(19) United States
(12) Patent Application Publication
(10) Pub. No.: US 2011/0028981 A1
(43) Pub. Date: Feb. 3, 2011

(54) BONE GRAFT MEASURING APPARATUS AND METHOD OF USE

(75) Inventor: William F. MCKAY, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/511,585

(22) Filed: Jul. 29, 2009

(51) Int. Cl. A61B 17/58; A61M 29/00

(52) U.S. Cl. 606/102; 606/192

(57) ABSTRACT

A bone void measuring apparatus includes a compliant inflatable member adapted for disposal with bone adjacent soft tissue. The bone has a bone defect surface that defines a bone defect cavity. A lumen is connected to the inflatable member and a fluid source for communication therebetween. The inflatable member receives a fluid of the fluid source for compliant inflation in a configuration such that outer surface of the inflatable member engagingly conforms to the bone defect surface and avoids displacement of the adjacent soft tissue to measure a volume of the bone defect cavity. The measured volume of the bone defect cavity is equivalent to a volume of bone void filler configured for introduction to the bone defect cavity. Methods of use are also disclosed.

### TECHNICAL FIELD

The present disclosure generally relates to the treatment of bone defects, and more particularly to methods and apparatus for assessing a volume of a bone defect cavity to determine a corresponding volume of bone filler material for disposal with the bone defect cavity.

### BACKGROUND OF THE INVENTION

Any publications or references discussed herein are presented to describe the background of the invention and to provide additional detail regarding its practice. Nothing herein is to be construed as an admission that the inventors are not entitled to antedate such disclosure by virtue of prior invention.

Bone defects can occur as a result of various conditions including bone damage, tumor, infection and degeneration. Such bone defects can include void space created within a bone that prevents or impairs normal body function. If not healed properly, these bone defects can pose a significant health risk to an individual.

Bone defects can be repaired with any number of bone void filler material such as bone graft filler, which may demonstrate osteogenesis, osteoinduction and/or osteoconduction for the purpose of regenerating bone or non-regenerative bone void fillers, such as Poly(methyl methacrylate) (PMMA) based bone cements. Bone graft material is implanted in the bone defect to fill the void space and grow new bone, thus allowing the bone to heal and in doing so, restoring body function. Bone grafts may include, for example, crushed bone (cancellous and cortical), or a combination of these (and/or other natural materials) and synthetic biocompatible materials intended to stimulate growth of healthy bone. Alternatively, a void may be filled with a settable polymer, such as a PMMA based bone cement, that does not stimulate bone growth but instead provides its own structural stabilizing ability.

In a known bone void filling procedure, a surgeon packs a bone defect until the bone void filling material extrudes therefrom. This procedure requires excess bone void filling material in that the volume of bone void filling material is larger than the bone defect such that the bone defect is overfilled. This can cause an over-concentration of growth factor in the affected area, such as, for example, in applications employing a compressible carrier such as a collagen sponge. This drawback can expose the affected area to adverse events. Conversely, if the volume of bone void filling material is less than the volume of the bone defect, the bone defect will not be adequately filled, and new bone will not grow or will not grow to completely fill the bone defect.

Employing an accurate amount of bone void filling material can affect the success of other applications such as intervertebral implants. In these applications, osteogenic material is packed around an implant in the intervertebral space to promote fusion between adjacent vertebrae. However, if the physician is not able to adequately view the amount of osteogenic material being packed around the implant, or if the osteogenic material is not tightly bound together, portions of the osteogenic material may fall out during packing. This can disadvantageously lead to osteogenic debris floating within the patient, which can lead to complications and consequent health risk. Accordingly, it is desirable to assess the volume of a bone defect to determine an accurate volume of bone graft material required for a procedure.

Various attempts have been made to accurately determine the volume associated with a bone defect. However, the concealed location and/or irregular shape of the bone defect make it difficult to assess an accurate volume and/or size of the bone defect. Known bone defect sizing techniques include imaging tests such as X-ray, MRI and CAT, highly invasive procedures such as open surgery and intraoperative measures such as molds, laser and ultrasound. These attempts at bone defect sizing, however, can disadvantageously pose a health risk to a patient, involve complex and expensive equipment, and/or perform inaccurate measurement of dimension and/or configuration.

Therefore, it would be desirable to provide a minimally invasive apparatus and method for assessing a volume of a bone defect cavity to determine a corresponding volume of bone void fillermaterial for disposal within the bone defect cavity. Desirably, such an apparatus and method provides a compliant, low pressure inflatable member for measuring volume. It would be most desirable if the inflatable member is configured to engagingly conform to various bone defect surfaces.

### SUMMARY OF THE INVENTION

Accordingly, the present disclosure provides an apparatus and method for assessing a volume of a bone defect cavity to determine a corresponding volume of bone filling material for disposal with the bone defect cavity. Desirably, the apparatus and method provide a compliant, low pressure inflatable member for measuring the volume of a bone defect cavity. It would be most desirable if the inflatable member is highly compliant to allow the inflatable member to engagingly conform to various irregular bone defect surfaces.

In one embodiment of the present disclosure, an instrument is provided for assessing the volume of a bone defect prior to implantation of a bone filler material. In another embodiment, the invention provides a method wherein the volume of a bone void is determined by inserting a compliant extendable member in a deflated condition within the bone void area, wherein the extendable member is connected to a liquid or gas source having a measurement capability, inflating the compliant extendable member with a measured amount of the liquid or gas under low pressure, determining the void volume based on the measurement of the liquid or gas volume, withdrawing the liquid or gas from the compliant extendable member and thereby returning it to its deflated condition, and removing the extendable member from the bone void area.

In another embodiment, the apparatus of the present disclosure includes an extremely soft and/or compliant balloon at a first end of a cannula that is easily inflated with fluid such as saline or air. The balloon can be inflated with very minimal pressure to measure void space, while avoiding the displacement of adjacent soft tissues and without crushing, further fracturing and/or compressing the surrounding bone. The apparatus can be employed with a material to measure an accurate void volume of the bone defect. In an exemplary embodiment, the apparatus and/or balloon may be disposable. In another embodiment, a plastic syringe may be connected to a first end of a cannula and used to inflate a balloon disposed at the other end of the cannula. In yet another exemplary embodiment, the apparatus is included in a bone graft or bone filler kit and ready to use. The apparatus may be employed such that a surgeon employs the kit with a method, which includes the step of inserting the un-inflated balloon into a bone defect. The balloon is inflated with minimal pressure with a plunger of the syringe. The volume displaced out of the syringe into the balloon is recorded and the amount of bone filling material is determined based on the recorded volume. The surgeon may then deflate the balloon by retracting the syringe plunger and the balloon may be easily removed from the subject. The surgeon then inserts the appropriate amount of bone filling material into the defect cavity.

The apparatus may be employed such that the method not only measures the volume of a bone void or bone defect, but also measures the volume of a cannula, or any other fluid channel path in connection with the bone void or bone defect and the delivery source for the bone filling material, such that a surgeon can provide an exact amount of bone void filler (bone filling material) through the empty cannula into the bone void or defect. Hence, in this embodiment the liquid is channeled through the cannula into the balloon, with the liquid starting at a position approximately equivalent to the position from which the bone void filler is going to enter the cannula, and after measurement of the bone void or defect volume, along with the cannula volume, the liquid is completely removed. For example, the balloon is inflated with minimal pressure by injecting a liquid from a syringe connected to the cannula and the volume displaced out of the syringe into the balloon is recorded, the liquid is removed by withdrawing the plunger to at least its original position and the bone void filler, which may optionally also be contained in a measured syringe, is connected to the cannula and the measured amount of the bone void filler is injected into the cannula and bone defect cavity.

The apparatus and method of the present disclosure may be advantageously employed when using a bone growth factor in or near cancellous bone, which has a high surface area due to the interconnected spaces that would typically be occupied by bone marrow. Examples of surgical procedures where cancellous bone may be present include interbody fusions, or proximal and distal femur fracture applications. The apparatus and method of the present disclosure may also be employed with bone defects and/or medical procedures such as, Alveolar ridge augmentation, sinus lift procedures, apicectomy procedures, bone cyst resection, vascular necrosis and acetabular defects. Such an apparatus and/or method is also useful to ensure an adequate volume of bone void filler is used, such that the bone defect is not under and/or over filled, such as in sinus elevation procedures and long bone defects.

In one particular embodiment, in accordance with the principles of the present disclosure a bone void filler measuring apparatus may also be provided. The bone void or bone cavity measuring apparatus includes a compliant inflatable member adapted for disposal within bone that is adjacent to soft tissue. The bone has a bone defect surface that defines a bone defect cavity. A lumen is connected to the inflatable member and a fluid source for communication therebetween. The inflatable member receives a fluid from the fluid source for compliant inflation in a configuration such that the outer surface of the inflatable member engagingly conforms to the bone defect surface and avoids displacement of the adjacent soft tissue, thereby measuring the volume of the bone defect cavity. In this example, the balloon may have a plurality of regions with each region having a different modulus of elasticity, such that a first region is made of a highly compliant material and a second region is made of a firmer and less compliant material, such that one surface of the balloon does not expand under operational pressures, thereby preventing displacement of the surrounding soft tissue, and a second surface that rapidly expands in response to low pressure to measure the bone defect cavity. The measured volume of the bone defect cavity is then used to deliver a measured amount of a bone void filler into the bone defect cavity by way of a bone void filler measuring apparatus.

The bone defect surface may include at least one implant adapted for disposal within the bone defect cavity.

The inflatable member can be a low pressure balloon. The inflatable member is configured for compliant inflation within a cavity of the implant such that the outer surface of the inflatable member engagingly conforms thereto to measure a volume of the cavity. The inflatable member can be configured for insertion within an intervertebral cavity of the bone defect. The inflatable member may also be configured for insertion within a femur fracture of the bone defect surface.

The fluid may include at least one medical imaging contrast agent. The bone defect can be an intervertebral cavity and may include at least one implant or be receiving an implant. The fluid source can be a syringe connected to the lumen or any other device or pump capable of extruding and retracting a measurable amount of a fluid. The syringe, device or pump being configured to measure the volume of the bone defect cavity by way of filling a lumen in the inflatable member (e.g., a balloon).

In an alternate embodiment, a surgical bone void filler kit is provided that includes a bone defect measuring apparatus, as described herein, and an instrument assembly for delivering a predetermined amount of bone void filler material based on the measured volume of the bone defect cavity. In another embodiment, the bone void filler is a bone cement, for example, a PMMA based bone cement, wherein extrusion of the bone void filler material is considered to be undesirable.

In another alternate embodiment, a method for determining a volume of bone void filler for introduction to a bone defect cavity is provided. The method includes the steps of: providing a compliant inflatable member having a lumen and adapted for disposal within a bone defect that is adjacent to soft tissue, the inflatable member having at least two regions wherein a first region has a compliancy higher than a second region, such that the inflatable member expands asymmetrically in response to increasing pressure, with the first region expanding into the bone defect area and the second region resisting expansion and preventing displacement of the adjacent soft tissue, the lumen of the inflatable member being connected to a fluid source for communication therebetween, wherein the inflatable member receives a fluid from the fluid source for compliant inflation in a configuration such that an outer surface of the second region of the inflatable member engagingly conforms to the bone defect surface and avoids displacement of the adjacent soft tissue and/or bone to measure a volume of the bone defect cavity; inserting the inflatable member into the bone defect cavity; inflating the inflatable member with the fluid for compliant inflation such that the outer surface of the inflatable member engagingly conforms to the bone defect surface to measure a volume of the bone defect cavity; measuring the volume of the bone defect cavity; formulating a volume of bone void filler material based on the measured volume of the bone defect cavity; and injecting a measured amount of the bone void filler material.

### BRIEF DESCRIPTION OF THE DRAWINGS

The features of the present disclosure will become more readily apparent from the specific description accompanied by the following drawings, in which:

FIG. 1 is a perspective view of one particular embodiment of a bone graft measuring apparatus in accordance with the principles of the present disclosure;

FIG. 2 is a perspective view of another embodiment of the bone graft measuring apparatus of the invention wherein the fluid measuring device is illustrated as a syringe and the inflatable member is illustrated on the distal end of a cannula in the uninflated state;

FIG. 3 is a perspective view of another embodiment of the bone graft measuring apparatus of the invention wherein the fluid measuring device is illustrated as a syringe and the inflatable member is illustrated on the distal end of a cannula in the inflated state;

FIG. 4 is a bottom view of an intervertebral cavity with the bone graft measuring apparatus shown in FIG. 1 disposed therein in an uninflated orientation;

FIG. 5 is a bottom view of the intervertebral cavity shown in FIG. 4 with the bone graft measuring apparatus shown in FIG. 1 disposed therein in an inflated orientation;

FIG. 6 is a side view of a bone defect including an implant having the bone graft measuring apparatus shown in FIG. 1 disposed therein in an uninflated orientation;

FIG. 7 is a side view of the bone defect shown in FIG. 6 including the implant having the bone graft measuring apparatus shown in FIG. 1 disposed therein in an inflated orientation;

FIG. 8 is a side view of vertebrae including a bone defect with the bone graft measuring apparatus shown in FIG. 1 disposed therein in an inflated orientation;

FIG. 9 is a side view of a femur including a bone defect with the bone graft measuring apparatus shown in FIG. 1 disposed therein in an inflated orientation;

FIG. 10 is a side view of a mandible including a bone defect with the bone graft measuring apparatus shown in FIG. 1 disposed therein in an inflated orientation; and

FIG. 11 is a side view of a bone defect including a bone cyst area with the bone void measuring apparatus shown in FIG. 1 disposed therein in an inflated orientation.

Like reference numerals indicate similar parts throughout the figures.

### DETAILED DESCRIPTION OF THE INVENTION

As used herein, “compliant” or “compliance” means a measure of the ease with which the inflatable member (e.g., the balloon) may be deformed or distended in response to a pressure of less than 50 psi, less than 40 psi, less than 30 psi, less than 20 psi, less than 10 psi, less than 5 psi or less than 2 psi, and wherein small variations in pressure within the inflatable member cause a large change in the volume of the inflatable member, for example, an inflatable member having a diameter of about 1 mm at about 1 psi will expand to a diameter of at least about 3 mm at about 2 psi or less.

As used herein, “bone void filler” or “bone void filling material” means any substance adapted for insertion into, around or between a bone and includes, but is not limited to, bone morphogenic proteins and any related carrier, calcium phosphate based compositions, collagen based compositions, demineralized bone matrix based compositions, thermoplastic based compostions, such as poly(methyl methacrylate) based bone cements, and combinations thereof.

As used herein, “bone defect cavity” means any crack, cavity, necrotic area or gap in a bone or between two bones, for example, a bone defect cavity may be an intervertebral disc space or a compression fracture in a vertebrae.

Also, as used herein, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “having” “containing,” “characterized by,” and grammatical equivalents thereof are inclusive or open-ended terms that do not exclude additional, unrecited elements or method steps, but it will also be understood to include the more restrictive terms “consisting of” and “consisting essentially of.”

The exemplary embodiments of the apparatus and methods of the subject invention are discussed in terms of medical devices and methods for the treatment of bone defects and more particularly, in terms of an apparatus and method for assessing a volume of a bone defect cavity to determine a corresponding volume of bone void filler material for disposal with the bone defect cavity. It is contemplated that the apparatus includes an inflatable member that engagingly conforms to a bone defect surface to measure a volume thereof corresponding to a required amount of bone void filler material used for bone defect repair. It is envisioned that the present disclosure may be employed to treat bone defects in or near cancellous bone such as, for example, interbody fusions, and proximal and distal femur fracture applications. It is further envisioned that the present disclosure may be employed with bone defect applications such as alveolar ridge augmentation, sinus lift, tooth extraction, apicectomy, bone cyst, vascular necrosis, acetabular defects, sinus elevation procedures and long bone defects. The present disclosure can be used with surgical treatments including open surgery and minimally invasive procedures.

It is contemplated that the present disclosure may be employed with other osteal and bone related applications, including those associated with diagnostics and therapeutics. It is further contemplated that the apparatus and methods of the subject invention may be employed in a surgical treatment with a patient in a prone or supine position, employing a posterior, lateral or anterior approach. The present disclosure may be employed with procedures for treating the lumbar, cervical, thoracic and pelvic regions of a spinal column. Bone defects, as discussed herein, include fractures, bone voids and other areas of bone structures having discontinuities, cavities, recesses or the like (all of which are included within the meaning of “bone defect cavity” as used herein).

The present invention may be understood more readily by reference to the following detailed description of the invention taken in connection with the accompanying figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

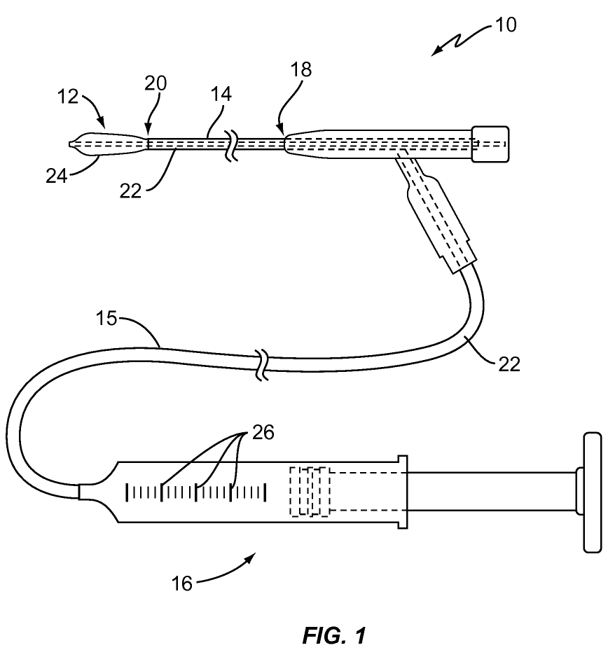

The following discussion includes a description of the apparatus of the subject invention, and related components and exemplary methods of employing the apparatus of the subject invention. Alternate embodiments are also disclosed. Reference will now be made in detail to an exemplary embodiment of the present disclosure, which is illustrated in the accompanying figures. Turning now to FIG. 1, the components of a bone graft measuring apparatus 10 and related methods of use, in accordance with the principles of the present disclosure, are described.

The components of the bone graft measuring apparatus 10 can be generally fabricated from materials suitable for medical applications, including metals, polymers, ceramics, biocompatible materials and/or their composites, and combinations thereof, depending on the particular application and/or preference of a medical practitioner. The components of apparatus 10 may include radiolucent and/or radio opaque materials.

Bone graft measuring apparatus 10 includes a compliant, low pressure inflatable member, such as, for example, a balloon 12. Balloon 12 is adapted for disposal within a bone defect that is adjacent to soft tissue, as will be discussed in further detail below. It is contemplated that the bone area has a bone defect surface that defines a bone defect cavity, the volume of which will be measured by the bone graft measuring apparatus 10 to determine a volume of bone graft or bone filler material required for effectively repairing the bone defect.

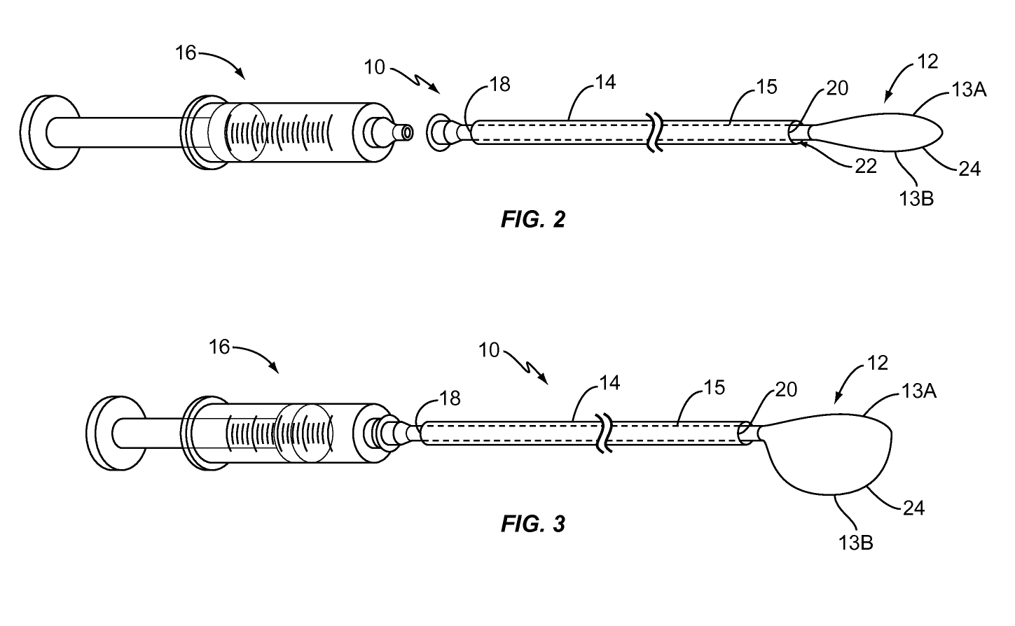

Balloon 12 is configured for disposal within a bone defect cavity and is expandable and can be contracted, between a non-expanded, non-inflated configuration, as shown in FIG. 2, and an expanded, inflated configuration, as shown in FIG. 3. It is contemplated that the inflatable member may include alternate or combinations of an expanding structure such as balloons, expanding arms, flexible wire, expanding linkages, tongs, expanding bands and articulating linkages. The balloon 12 shown in FIGS. 1 and 2 comprises two regions 13A and 13B, wherein region 13A is substantially resistant to expansion under low pressure and region 13B is highly flexible and expandable under low pressure. In this embodiment the measuring apparatus 10 may be inserted into a patient such that balloon 12 is placed between soft tissue surrounding a bone and over a bone defect cavity, upon inflation of the balloon 12 region 13B will expand into the bone defect cavity and region 13A may form a flexible guide or cap between the bone surface and the surrounding soft tissue.

Balloon 12 may be made of various polymeric materials such as polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof.

It is envisioned that balloon 12 may also be fabricated from biologically acceptable materials including vinyl, polyvinyl chloride, silicone, nylon, thermoplastic rubbers, thermoplastic elastomer materials, polyethylenes, ionomer, polyurethane, polyolefins, polyetheretherketone, polyactide, polyglyclolide, poly(lactide-co-glycolide), poly(dioxanone), poly (c-caprolactone), poly(hydroxylbutyrate), poly(hydroxylvalerate), tyrosine-based polycarbonate, polypropylene fumarate, polyethylene tetraphthalates (PET), or combinations thereof. Balloon 12 may be constructed of materials to achieve various desired characteristics such as biocompatibility, strength, thickness, elasticity, compliancy, durability and permeability.

It is contemplated that balloon 12 is capable of withstanding a tensile stress of greater than about 15 pounds without tearing, rupturing, or separating from the component parts of apparatus 10 when disposed in a bone defect cavity. Preferably, balloon 12 withstands a tensile stress of greater than about 10 pounds and expands to a volume of at least about 15 cm³ without tearing such that balloon 12 is readily inflatable with a small amount of inflation force and capable of stretching to engagingly conform to a bone defect surface. Preferably, balloon 12 is capable of withstanding 30 cm³ volume of inflation in an unconfined state without bursting.

It is contemplated that balloon 12 is capable of being fully expanded with an internal pressure of less than about 0.01 to 80 psi, less than about 0.5 to 50 psi, less than about 0.5 to 2 psi and less than about 1 to 10 psi when disposed in a bone defect cavity. The balloon 12 of the invention has a high degree of elasticity such that small variations in pressure cause wide variations in balloon diameter. For example, to achieve 50% elongation, a pressure of less than about 80 psi may be required, a pressure of less than about 50 psi may be required, a pressure of less than about 30 psi may be required, a pressure of less than about 10 psi may be required, a pressure of less than about 5 psi may be required or a pressure of less than about 1 psi may be required.

It is also contemplated that balloon 12 is made of a material having a high modulus of elasticity, such that the balloon 12 can conform to a highly convoluted path, such as found in a bone defect site.

It is contemplated that the inflatable member may be filled with image contrast agents and/or radioactive materials, and as such it is preferred to fabricate the inflatable member from chemical-resistant materials. The inflatable member may also be made from a multi-layered material with an inner expandable chemically resistant layer, and/or the interior of the inflatable member may be coated with a chemically resistant coating.

A fluid communication channel, such as, for example, a cannula 14 and/or flexible conduit 15 is connected to a lumen in the balloon 12 and a fluid source, such as, for example, a fluid reservoir and fluid displacement measuring device 16 for communication therebetween.

Balloon 12 and a distal portion of cannula 14 and/or flexible conduit 15 are configured for insertion through an entry site of a patient (not shown). It is contemplated that apparatus 10 in the description and claims of the present disclosure may be used in combination with other devices, such as cannulas, trocars, sheaths, minimally invasive instruments and the like. It is envisioned that cannula 14 and/or flexible conduit 15 can vary in length and cross section according to the requirements of a particular application. A proximal end 18 and a distal end 20 of the cannula 14 and/or flexible conduit 15 may have uniform or alternate cross section sizes and geometries. It is envisioned that cannula 14 and/or flexible conduit 15 or portions thereof may be flexible, semi-rigid, rigid and/or combinations thereof.

Fluid reservoir and fluid displacement measuring device 16 may be connected to the proximal end 18 of cannula 14 and/or flexible conduit 15. Fluid reservoir and fluid displacement measuring device 16 is configured to be capable of holding a fluid and adapted to be connected to and in fluid communication with the balloon 12 through and/or by way of the flexible conduit 15, wherein the fluid communication may be by way of a passageway 22 for delivery of the fluid to the balloon 12. As such, fluid reservoir and fluid displacement measuring device 16 delivers a measured amount of fluid to the balloon 12. Fluid reservoir and fluid displacement measuring device 16 may include graduated markings 26 or other indicia to measure the volume of fluid delivered to balloon 12 to determine the volume of a bone defect. It is contemplated that fluid reservoir and fluid displacement measuring device 16 may also provide volume measurement and appropriate readout via other methods know in the art.

It is envisioned that the fluid reservoir and fluid displacement measuring device 16, cannula 14 and/or flexible conduit 15 and balloon 12 may be connected in a monolithic formation, integral connection, and/or may include various complementary connection mechanisms such as, for example, a luer lock connector, puncture seal, insertion seal, pressure fit, and/or clips/clamps.

The cross section of passageway 22 can vary in size and geometry according to the requirements of a particular application. It is envisioned that the fluid source may alternatively be a pressurized container and/or wall connection. The flow of inflating fluid may be regulated and/or valve controlled manually, electronically or processor controlled, as is known to one skilled in the art. It is envisioned that cannula 14 may include one or a plurality of conduits through which inflating fluid is introduced to balloon 12. It is further envisioned that the fluid is introduced at a pressure in the range of between about 80 psi and 0 psi, between about 50 psi and about 0.1 psi, between about 30 psi and about 0.1 psi, between about 10 psi and about 0.1 psi, between about 5 psi and about 0.1 psi, between about 0.5 psi and about 2 psi or between about 1 psi and about 0.01 psi. The pressurized flow may be constant or varying, depending on the application.

Fluid reservoir and fluid displacement measuring device 16 delivers a low pressure fluid or medium, such as, for example, inflating air, gas, or other fluid that is introduced into balloon 12 such that balloon 12 assumes its inflated configuration. It is contemplated that alternative volume measuring mediums may be employed such as sterile water or saline. Balloon 12 receives a volume measuring fluid from fluid reservoir and fluid displacement measuring device 16 for compliant inflation in a configuration such that an outer surface 24 of balloon 12 engagingly conforms to the bone defect surface. This configuration avoids displacement of any soft tissue adjacent to the bone defect to accurately measure a volume of a bone defect cavity. As such, the measured volume of the bone defect cavity is used to determine a required volume of bone void filler material to repair the bone defect cavity, preferably without the bone void filler material being extruded from the bone defect site, or without the need to harvest unnecessary autograft material.

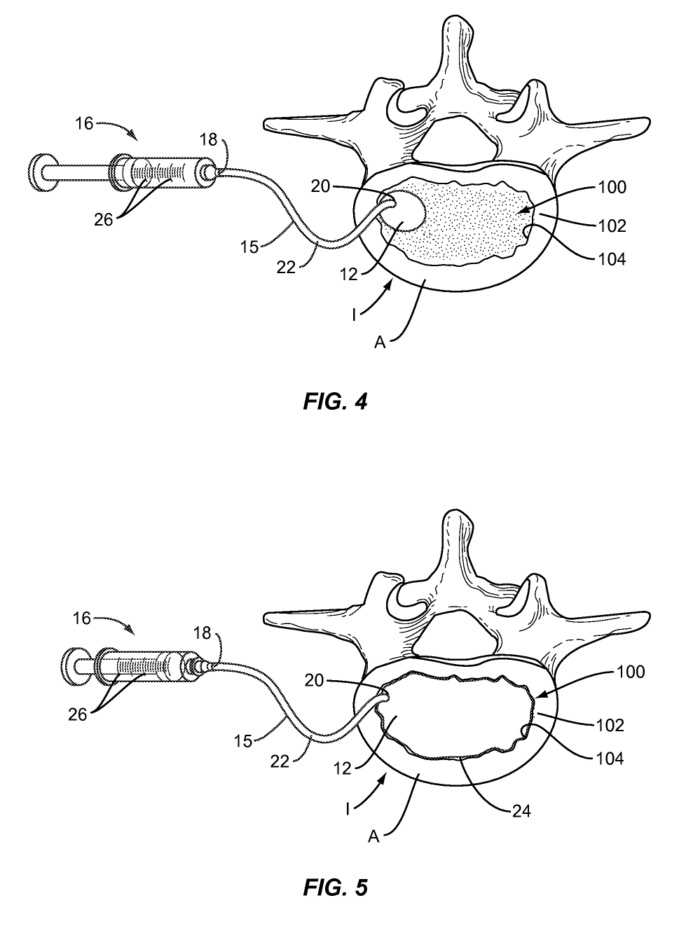

In another embodiment, the bone graft measuring apparatus 10 is a component of a surgical bone repair kit, which also includes the bone graft measuring apparatus 10, a minimally invasive instrument assembly for delivering the components of the kit and bone void filler material. For example, in one embodiment, bone graft measuring apparatus 10, similar to that described, is employed to determine a volume of bone void filler material based on measuring a volume of a bone defect cavity, as shown in FIGS. 4 and 5, in accordance with the principles of the present disclosure. The method may include or form parts of a surgical procedure, as described herein, or alternatively be used with diagnostic or other treatment procedures.

As part of a surgical procedure and/or separate treatment in contemplation of surgery, the distal end 20 of cannula 14 is introduced at or near a bone defect area in a patient's body via an instrument assembly, which includes a minimally invasive portal that provides access to the bone defect from a position external to the patient's body, as is known to one skilled in the art. The instrument assembly is introduced into the soft tissue of a patient, adjacent to the bone having a bone defect surface, such as, for example, intervertebral disc I. Intervertebral disc I having a bone defect cavity, such as, for example, a cavity 100 within the intervertebral disc I or alternatively within a verterbral body. To measure the volume of the cavity 100, the bone void measuring device 10 is inserted through the cannula 14 and the balloon 12 located at the distal end 20 is inflated to fill the bone defect cavity 100, and the volume of fluid or air used needed to expand the balloon 12 is used to calculate the bone defect cavity volume.

Visualization of the bone defect area may be accomplished using a remote viewing device, such as a fluoroscope or x-ray device, as conventionally utilized in minimally invasive procedures. Upon visualization of the bone defect area, balloon 12 is inserted within the cavity of annulus fibrosus A, as shown in FIG. 4, via the instrument assembly (not shown). The fluid reservoir and fluid displacement measuring device 16 contains an initial amount/volume of fluid/medium, which is measured and recorded via indicia of fluid reservoir and fluid displacement measuring device 16. Balloon 12 is inflated with fluid/medium such as air, biologically compatible gas or fluid via cannula 14 and fluid reservoir and fluid displacement measuring device 16, such that balloon 12 assumes its expanded, inflated configuration, as shown in FIG. 5.

Balloon 12 engages surrounding disc tissue 102 including an interior wall 104 of annulus fibrosus A such that outer surface 24 of balloon 12 engagingly conforms to interior wall 104 of annulus fibrosus A and avoids displacement of interior wall 104. Subsequent to inflation of balloon 12 via syringe 16, the remaining amount/volume of fluid contained within syringe 16 is measured and recorded. This configuration of the present disclosure facilitates an accurate measurement of the volume of cavity 24 of the damaged annulus fibrosus A. By avoiding displacement of interior wall 104, inaccurate volume measurements of bone defect cavity 100 are avoided. The volume of cavity 100 is determined using the recorded volume measurements of syringe 16, as discussed, due to the inflation of balloon 12 and its engaging conformity with interior wall 104. The measured volume of cavity 100 is used to determine and/or formulate a required amount of bone graft material that will be introduced within cavity 100 and employed to repair intervertebral disc I.

Bone graft measuring apparatus 10 may be employed to take one or a plurality of volume determinations to confirm accuracy or measure other cavities. Upon completion, fluid reservoir and fluid displacement measuring device 16 is manipulated to withdraw the fluid from balloon 12 such that balloon 12 contracts and assumes its uninflated configuration. Balloon 12 is withdrawn from annulus fibrosus A and bone graft measuring apparatus 10 is withdrawn from the patient.

After determination and/or formulation of the required amount/volume of bone void filler material for repair of intervertebral disc I, the bone void filler material is prepared for delivery to cavity 100 of intervertebral disc I. Cavity 100 of intervertebral disc I is supplied with the bone void filler material delivered via a bone graft needle or other appropriate device (not shown), as is known to one skilled in the art. See, for example, that described in U.S. Pat. Nos. 4,501,269, 4,878,915, 4,961,740, 5,015,247, and U.S. Patent Publication No. 2005/0192669, the entire contents of each of these references being incorporated by reference herein.

The bone void filler material delivered to the bone defect area may be any synthetic or tissue-based material that optionally promotes bone growth and may be provided in paste form. The bone void filler material may be supplied to the bone graft needle or cannula via a conventional syringe. Once cavity 100 of intervertebral disc I has been supplied with the amount of bone graft material, as determined in accordance with the present disclosure, the bone graft needle is removed from the patient's body through the portal. The bone graft material remains in the patient's body to promote bone growth or regeneration.

During the spinal fusion procedure, similar to that described herein or in other procedures and/or treatments of bone defects within the contemplation of the present disclosure, the surgeon may apply an osteogenic material to bone defect cavity 100, or implant/fusion device (discussed with regard to FIGS. 4 and 5) by packing cavity 100 with an osteogenic material. Any suitable osteogenic material or composition may be used. Further, any suitable carrier that provides a vehicle for introducing the osteogenic material into the pores of the bone in growth material or the hollow interior of an implant device is contemplated. Such carriers are well known and commercially available. The choice of carrier material can be based on biocompatibility, biodegradability, mechanical properties and interface properties. The carrier may be any suitable carrier capable of delivering the proteins to the bone defect area, including for example open cell polylactic acid polymers (OPLA), biodegradable and chemically defined calcium sulfate, tricalcium phosphate (TCP), hydroxyapatite (HA), biphasic TCP/HA ceramic, polylactic acids and polyanhydrides. Other potential materials can be biodegradable and biologically well defined, such as bone or dermal collagen, pure proteins or extracellular matrix components. The osteoinductive material may also be a mixture of the osteoinductive cytokine and a polymeric acrylic ester carrier.

In an alternate embodiment, the fluid delivered to balloon 12 in connection with measuring the volume of the bone defect cavity may include one or a plurality of medical imaging contrast media. For example, the fluid may be selected from saline solutions and imaging contrast mediums, if it is desired to also image the bone defect area while the inflatable member is inflated therein. The imaging contrast media contemplated for use includes all applicable imaging contrast media, including contrast agents for X-ray, MRI imaging contrast media, CT (computerized tomography) and C-arm fluoroscopy media, and PET (positron emission tomography) imaging media.

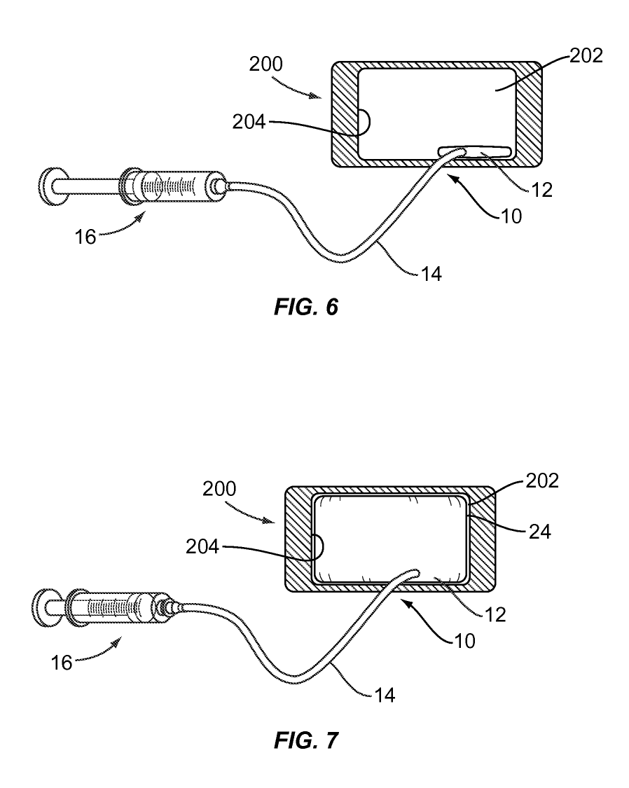

In another embodiment, as shown in FIGS. 6 and 7, bone graft measuring apparatus 10, similar to that discussed above, is employed with a method for determining a volume of bone void filler for introduction to a bone defect cavity whereby the bone defect surface includes one or a plurality of implants(s) 200. Implant 200 is adapted for disposal within a bone defect cavity, such as, for example, cavity 100 of intervertebral disc I discussed with regard to FIGS. 4 and 5. Balloon 12 is configured for compliant inflation within a cavity 202 of implant 200 such that outer surface 24 of balloon 12 engagingly conforms thereto to measure a volume of cavity 202.

The surgical bone graft kit discussed above is provided for a spinal fusion procedure employing implant 200. Distal end 20 of cannula 14 is introduced into the soft tissue of a patient adjacent intervertebral disc I, similar to that discussed with regard to FIGS. 4 and 5. Visualization of the bone defect area may be done by having balloon 12 inserted within cavity 202 of implant 200, as shown in FIG. 6, via the instrument assembly. An initial amount/volume of pressurized expanding fluid/medium is measured via indicia of fluid reservoir and fluid displacement measuring device 16. Balloon 12 is inflated with the fluid/medium via cannula 14 and fluid reservoir and fluid displacement measuring device 16, such that balloon 12 assumes its expanded inflated configuration, as shown in FIG. 7.

Balloon 12 engages the surrounding interior wall 204 of implant 200 such that outer surface 24 of balloon 12 engagingly conforms to interior wall 204. Subsequent to inflation of balloon 12 via fluid reservoir and fluid displacement measuring device 16, the remaining amount/volume of fluid contained within fluid reservoir and fluid displacement measuring device 16 is measured and recorded. The volume of cavity 202 is determined using the recorded volume measurements of fluid reservoir and fluid displacement measuring device 16, as discussed, due to the inflation of balloon 12 and its engaging conformity with interior wall 204. The measured volume of cavity 202 is used to determine and/or formulate a required amount of bone void filler material that will be introduced within cavity 202 and employed to repair intervertebral disc I with implant 200.

After determination and/or formulation of the required amount/volume of bone void filler material for repair of intervertebral disc I with implant 200, the bone graft material is prepared for delivery to cavity 202. Cavity 202 is supplied with bone void filler material, similar to that described above and is known to one skilled in the art.

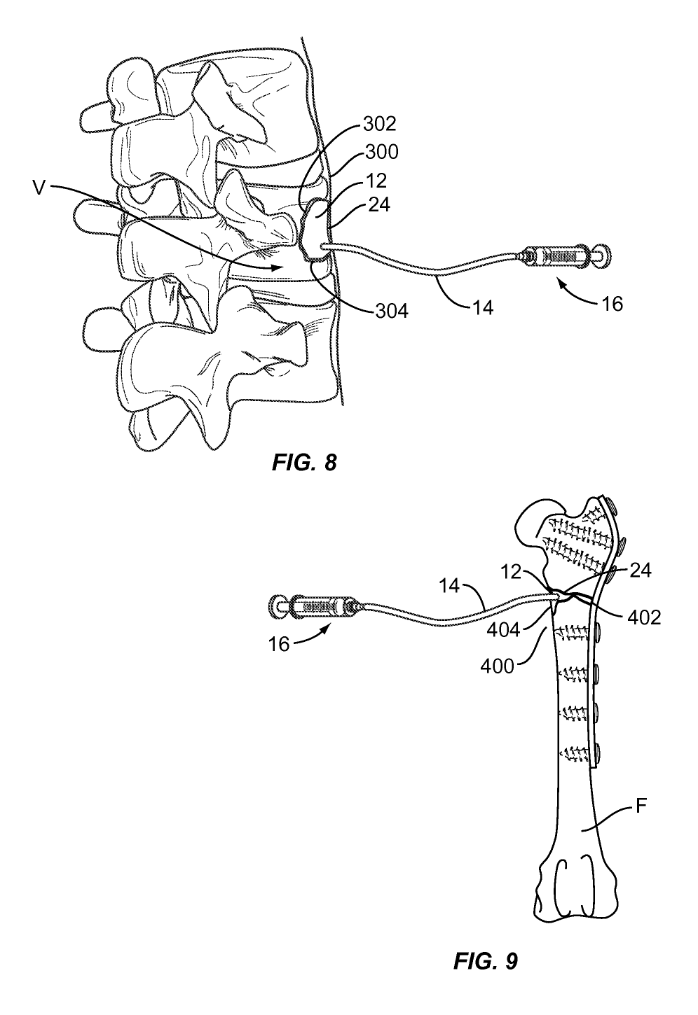

In another embodiment, as shown in FIG. 8, bone graft measuring apparatus 10 is employed with a method for determining a volume of a bone defect cavity whereby the bone defect surface includes a fractured vertebrae V. The surgical bone graft kit discussed above is provided for treatment of fractured vertebrae V. Distal end 20 of cannula 14 is introduced into soft tissue 300 of a patient adjacent fractured vertebrae V, similar to that discussed above. Upon visualization of the bone defect area, balloon 12 is inserted within cavity 302 of vertebrae V via the instrument assembly. An initial amount/volume of pressurized expanding fluid/medium is measured and recorded via indicia of fluid reservoir and fluid displacement measuring device 16. Balloon 12 is inflated with the pressurized expanding fluid/medium via cannula 14 and fluid reservoir and fluid displacement measuring device 16, such that balloon 12 assumes its expanded, inflated configuration, as shown in FIG. 8.

Balloon 12 engages surrounding soft tissue 300 and an interior wall 304 of cavity 302 such that outer surface 24 of balloon 12 engagingly conforms to interior wall 304 and avoids displacement of soft tissue 300. Subsequent to inflation of balloon 12 via fluid reservoir and fluid displacement measuring device 16, the remaining amount/volume of fluid contained within fluid reservoir and fluid displacement measuring device 16 is measured and recorded. The volume of cavity 302 is determined using the recorded volume measurements of fluid reservoir and fluid displacement measuring device 16, as discussed, due to the inflation of balloon 12 and its engaging conformity with interior wall 304. The measured volume of cavity 302 is used to determine and/or formulate a required amount of bone void filler material that will be introduced within cavity 302 and employed to repair vertebrae V.

After determination and/or formulation of the required amount/volume of bone void filler material for repair of fractured vertebrae V, the bone void filler material is prepared for delivery to cavity 302. Cavity 302 is supplied with the bone void filler material, similar to that described above and is known to one skilled in the art.

In another embodiment, as shown in FIG. 9, bone graft measuring apparatus 10 is employed with a method for determining a volume of a bone defect cavity whereby the bone defect surface includes a long bone fracture of a femur F. The surgical bone graft kit discussed above is provided for treatment of fractured femur F. Distal end 20 of cannula 14 is introduced into soft tissue 400 of a patient adjacent fractured femur F, similar to that discussed above. Upon visualization of the bone defect area, balloon 12 is inserted within a cavity 402 of femur F via the instrument assembly. An initial amount/volume of fluid/medium is measured and recorded via indicia of fluid reservoir and fluid displacement measuring device 16. Balloon 12 is inflated with pressurized fluid/medium via cannula 14 and fluid reservoir and fluid displacement measuring device 16, such that balloon 12 assumes its expanded, inflated configuration, as shown in FIG. 9.

Balloon 12 engages the surrounding soft tissue 400, including an interior wall 404 of cavity 402 such that outer surface 24 of balloon 12 engagingly conforms to interior wall 404 and avoids displacement of soft tissue 400. Subsequent to inflation of balloon 12 via fluid reservoir and fluid displacement measuring device 16, the remaining amount/volume of fluid contained within fluid reservoir and fluid displacement measuring device 16 is measured and recorded. The volume of cavity 402 is determined using the recorded volume measurements of fluid reservoir and fluid displacement measuring device 16 (the post fill volume is subtracted from the volume prior to filling the balloon 12), as discussed, due to the inflation of balloon 12 and its engaging conformity with interior wall 404. The measured volume of cavity 402 is used to determine and/or formulate a required amount of bone graft material that will be introduced within cavity 402 and employed to repair femur F.

After determination and/or formulation of the required amount/volume of bone void filler material for repair of fractured femur F, the bone void filler material is prepared for delivery to cavity 402. Cavity 402 is supplied with bone graft material, similar to that described above and is known to one skilled in the art.

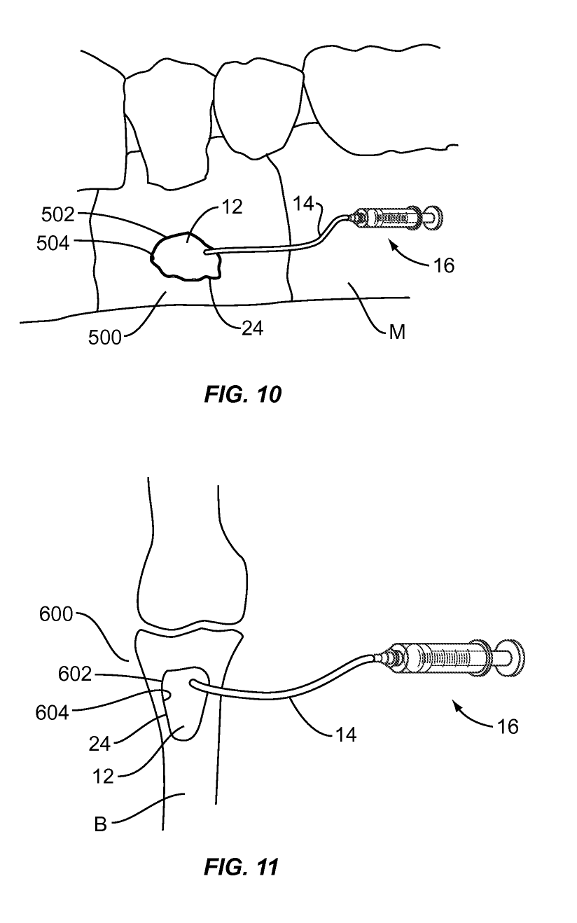

In another embodiment, as shown in FIG. 10, bone graft measuring apparatus 10 is employed with a method for determining a volume of bone void filler for introduction to a bone defect cavity whereby the bone defect surface includes a mandible M. The surgical bone graft kit discussed above is provided for treatment of a damaged mandible M, which may require tooth extraction. Distal end 20 of cannula 14 is introduced into soft tissue 500 of the patient adjacent damaged mandible M, similar to that discussed above. Upon visualization of the bone defect area, balloon 12 is inserted within a cavity 502 of mandible M via the instrument assembly. An initial amount/volume of pressurized fluid/medium is measured and recorded via indicia of fluid reservoir and fluid displacement measuring device 16. Balloon 12 is inflated with pressurized fluid/medium via cannula 14 and fluid reservoir and fluid displacement measuring device 16, such that balloon 12 assumes its expanded inflated configuration, as shown in FIG. 8.

Balloon 12 engages the surrounding soft tissue 500, and interior wall 504 of cavity 502 such that outer surface 24 of balloon 12 engagingly conforms to interior wall 504 and avoids displacement of soft tissue 500. Subsequent to inflation of balloon 12 via fluid reservoir and fluid displacement measuring device 16, the remaining amount/volume of fluid contained within fluid reservoir and fluid displacement measuring device 16 is measured and recorded. The volume of cavity 502 is determined using the recorded volume measurements of fluid reservoir and fluid displacement measuring device 16, as discussed, due to the inflation of balloon 12 and its engaging conformity with interior wall 504. The measured volume of cavity 502 is used to determine and/or formulate a required amount of bone void filler material that will be introduced within cavity 502 and employed to repair mandible M.

After determination and/or formulation of the required amount/volume of the bone void filler material for repair of damaged mandible M, the bone void filler material is prepared for delivery to cavity 502. Cavity 502 is supplied with the bone graft material, similar to that described above and is know to one skilled in the art.

In another embodiment, as shown in FIG. 9, bone graft measuring apparatus 10 is employed with a method for determining a volume of bone void filler for introduction to a bone defect cavity whereby the bone defect surface includes an area of bone B having a bone cyst. The surgical bone graft kit discussed above is provided for treatment of a bone cyst of bone B, similar to that discussed above. Upon visualization of the bone defect area, balloon 12 is inserted within a cavity 602 of bone B, via the instrument assembly. An initial amount/volume of fluid/medium is measured and recorded via indicia of fluid reservoir and fluid displacement measuring device 16. Balloon 12 is inflated with pressurized expanding fluid/medium via cannula 14 and fluid reservoir and fluid displacement measuring device 16, such that balloon 12 assumes its expanded inflated configuration, as shown in FIG. 11.

Balloon 12 engages the surrounding soft tissue 600, including an interior wall 604 of cavity 602 such that outer surface 24 of balloon 12 engagingly conforms to interior wall 604 and avoids displacement of soft tissue 600. Subsequent to inflation of balloon 12 via fluid reservoir and fluid displacement measuring device 16, the remaining amount/volume of fluid contained within fluid reservoir and fluid displacement measuring device 16 is measured and recorded. The volume of cavity 602 is determined using the recorded volume measurements of fluid reservoir and fluid displacement measuring device 16, as discussed, due to the inflation of balloon 12 and its engaging conformity with interior wall 604. The measured volume of cavity 602 is used to determine and/or formulate a required amount of bone graft material that will be introduced within cavity 602 and employed to repair bone B.

After determination and/or formulation of the required amount/volume of the bone void filler material for repair of bone B, the bone void filler material is prepared for delivery to cavity 602B. Cavity 602 is supplied with the bone void filler material, similar to that described above and is know to one skilled in the art.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto. In addition, all patents, patent publications or other publications referenced herein are incorporated by reference in their entirety.

What is claimed is:

1. A bone graft measuring apparatus comprising:
a compliant inflatable member adapted for disposal with bone adjacent soft tissue, the bone having a bone defect surface that defines a bone defect cavity; and
a lumen connected to the inflatable member and a fluid source for communication therebetween;
the inflatable member receives a fluid of the fluid source for compliant inflation in a configuration such that outer surface of the inflatable member engagingly conforms to the bone defect surface and avoids displacement of the adjacent soft tissue to measure a volume of the bone defect cavity,
wherein the measured volume of the bone defect cavity is equivalent to a volume of bone graft configured for introduction to the bone defect cavity.

2. A bone graft measuring apparatus according to claim 1, wherein the inflatable member is a low pressure balloon.

3. A bone graft measuring apparatus according to claim 2, wherein the balloon is fabricated from the group consisting of polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof.

4. A bone graft measuring apparatus according to claim 1, wherein the bone defect surface includes at least one implant adapted for disposal within the bone defect cavity, the inflatable member being configured for compliant inflation within a cavity of the implant, such that the outer surface of the inflatable member engagingly conforms thereto to measure a volume of the cavity of the implant.

5. A bone graft measuring apparatus according to claim 1, wherein the fluid includes at least one medical imaging contrast media.

6. A bone graft measuring apparatus according to claim 4, wherein the bone defect surface is an intervertebral cavity that includes the at least one implant.

7. A bone graft measuring apparatus according to claim 1, wherein the fluid source is a syringe connected to the lumen, the syringe being configured to measure the volume of the bone defect cavity.

8. A bone graft measuring apparatus according to claim 1, wherein the inflatable member is configured for disposal within an intervertebral cavity of the bone defect surface.

9. A bone graft measuring apparatus according to claim 1, wherein the inflatable member is configured for disposal within a femur fracture of the bone defect surface.

10. A surgical bone graft kit comprising:
a bone graft measuring apparatus including:
a compliant inflatable member adapted for disposal with bone adjacent soft tissue, the bone having a bone defect surface that defines a bone defect cavity,
a lumen configured to be connected to the inflatable member;
a fluid source configured to be connected to the compliant inflatable member through the lumen for communication therebetween, wherein an outer surface of the compliant inflatable member engagingly conforms to the bone defect surface upon inflation with the fluid form the fluid source,and avoids displacement of the adjacent soft tissue, to measure a volume of the bone defect cavity;
the fluid source including a syringe configured to be connected to the lumen, the syringe being configured to measure a volume of the fluid employed to inflate the inflatable member corresponding to the volume of the bone defect cavity;
an instrument assembly for delivering bone void filler material; and
a volume of bone void filler material.

11. A surgical bone graft kit according to claim 10, wherein the inflatable member is a low pressure balloon.

12. A surgical bone graft kit according to claim 11, wherein the balloon is expands by at least 50% with less than about 10 psi of pressure.

13. A surgical bone graft kit according to claim 12, wherein the inflatable member comprises a first region and a second region, wherein the first region is more compliant than the second region and wherein the first region expands at a pressure of less than about 10 psi and the second region does not.

14. A method for determining a volume of a bone defect cavity, the method comprising the steps of:
providing a compliant inflatable member adapted for disposal within a bone defect cavity, a lumen connected to the inflatable member and a fluid source for communication therebetween;
inserting the inflatable member into a bone defect cavity;
inflating the inflatable member by introducing a fluid from the fluid source for compliant inflation such that an outer surface of the inflatable member engagingly conforms to the bone defect surface to measure a volume of the bone defect cavity; and
determining the volume of the bone defect cavity.

15. The method for determining a volume of a bone defect cavity according to claim 14, wherein the bone defect surface includes at least one implant adapted for disposal within the bone defect cavity, the inflatable member being configured for compliant inflation within a cavity of the implant, such that the outer surface of the inflatable member engagingly conforms thereto to measure a volume of the cavity within the implant.

16. The method for determining a volume of a bone defect cavity according to claim 14, wherein the fluid includes at least one medical imaging contrast media.

17. The method for determining a volume of a bone defect cavity according to claim 14, further comprising the step of providing the fluid source including a syringe connected to the lumen, the syringe being configured to measure a volume of the fluid employed to inflate the inflatable member corresponding to the volume of the bone defect cavity.

18. The method for determining a volume of a bone defect cavity according to claim 14, wherein the inflatable member is configured for disposal within a femur fracture of the bone defect surface.

19. The method for determining a volume of a bone defect cavity according to claim 14, wherein the inflatable member is configured for disposal within an intervertebral cavity of the bone defect surface.
